(19) United States
(12) Patent Application Publication
(10) Pub. No.: US 2006/0199819 A1
(43) Pub. Date: Sep. 7, 2006

(54) Inhibition of cyclooxygenase-2 activity

(75) Inventors: Andrew J. Dannenberg, New York, NY (US); George Muller, Bridgewater, NJ (US)

Correspondence Address: JONES DAY, 222 EAST 41ST ST, NEW YORK, NY, 10017, US

(73) Assignee: Celgene Corporation

(21) Appl. No.: 11/429,300

(22) Filed: May 4, 2006

Related U.S. Application Data
(63) Continuation of application No. 10/680,606, filed on Oct. 7, 2003, now abandoned.
Application No. 10/680,606 is a continuation of application No. 09/823,057, filed on Mar. 30, 2001, now abandoned.
(60) Provisional application No. 60/193,981, filed on Mar. 31, 2000.

(51) Int. Cl. A61K 31/5377; A61K 31/454

(52) U.S. Cl. 514/235.2; 514/323; 514/326

(57) ABSTRACT

The present invention provides new methods for inhibiting the activity of the enzyme cycloxygenase-2 (or COX-2). Inhibitors of COX-2 are known to be useful anti-inflammatory, analgesic and anti-angiogenic agents. The compounds in the present case are heterocyclic substituted 4-aminoglutarimides. Methods of using the compounds to inhibit prostaglandin synthesis are claimed.

This is a continuation of Ser. No. 09/823,057 filed on Mar. 30, 2001, which claims the benefit of U.S. Provisional Application No. 60/193,981 filed on Mar. 31, 2000 entitled Inhibition of Cyclooxygenase-2 Activity, hereby incorporated by reference into this application.

### FIELD OF THE INVENTION

The present invention pertains to methods for inhibiting the activity of the enzyme cyclooxygenase-2.

### BACKGROUND OF THE INVENTION

The components of angiogenesis relating to vascular endothelial cell proliferation, migration and invasion, have been found to be regulated in part by polypeptide growth factors. Endothelial cells exposed to a medium containing suitable growth factors can be induced to evoke some or all of the angiogenic responses. Polypeptides with in vitro endothelial growth promoting activity include acidic and basic fibroblast growth factors, transforming growth factors α and β, platelet-derived endothelial cell growth factor, granulocyte colony-stimulating factor, interleukin-8, hepatocyte growth factor, proliferin, vascular endothelial growth factor and placental growth factor. Folkman et al., 1995, N. Engl. J. Med., 333:1757-1763.

Inhibitory influences predominate in the naturally occurring balance between endogenous stimulators and inhibitors of angiogenesis. Rastinejad et al., 1989, Cell 56:345-355. In those instances in which neovascularization occurs under normal physiological conditions, such as wound healing, organ regeneration, embryonic development, and female reproductive processes, angiogenesis is stringently regulated and spatially and temporally delimited. Under conditions of pathological angiogenesis such as that characterizing solid tumor growth, these regulatory controls fail.

Various cell types of the body can be transformed into benign or malignant tumor cells. The most frequent tumor site is lung, followed by colorectal, breast, prostate, bladder, pancreas, and then ovary. Other prevalent types of cancer include leukemia, central nervous system cancers, including brain cancer, melanoma, lymphoma, erythroleukemia, uterine cancer, and head and neck cancer.

Unregulated angiogenesis sustains progression of many neoplastic and non-neoplastic diseases including solid tumor growth and metastases. See, e.g., Moses et al., 1991, Biotech. 9:630-634; Folkman et al, 1995, N. Engl. J. Med, 333:1757-1763; Auerbach et al., 1985, J. Microvasc. Res. 29:401-411; Folkman, 1985, Advances in Cancer Research, eds. Klein and Weinhouse, Academic Press, New York, pp. 175-203; Patz, 1982, Am. J. Opthalmol. 94:715-743; Folkman et al, 1983, Science 221:719-725; and Folkman and Klagsbrun, 1987, Science 235:442-447.

### DETAILED DESCRIPTION

Cyclooxygenase-2, the rate-limiting enzyme in prostaglandin biosynthesis, is expressed in tumor associated macrophages. Because prostaglandins, notable PGE₂, are important mediators of inflammatory response and angiogenesis, inhibition of their biosynthesis can be used to combat these effects. Inhibition of the cyclooxygenase-2 protein by a test compound can be conveniently observed in cells in which induction of the protein has been induced by lipopolysaccharide (LPS). Thus it is known that LPS enhances cyclooxygenase-2 transcription and this effect thus can be used as convenient model for evaluating cyclooxygenase-2 inhibition.

It has now been discovered that the activity of cyclooxygenase-2 can be inhibited by certain amides and imides and that this effect causes a reduction in prostaglandin biosynthesis. This effect in turn produces, inter alia, an anti-inflammatory response, anti-angiogenesis, and antineoplastic effect.

The amide or imide that can be employed in the present invention include all of those described in U.S. Pat. Nos. 2,830,991, 5,385,901, 5,635,517, 5,798,368, and 5,874,448, in PCT WO98/54170, and in Ser. No. 09/270,411 filed Mar. 16, 1999, the disclosure of each being incorporated herein by reference.

In particular, the amides and imides include compounds of the formula: 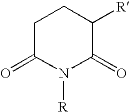
which R is hydrogen, alkyl of 1 to 6 carbon atoms, alkenyl of 2 to 6 carbon atoms, morpholinomethyl, phenyl, or benzyl, and

R′ is:

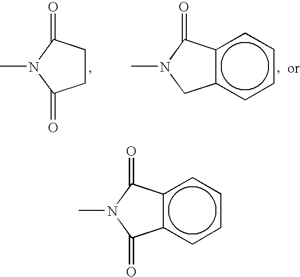

In one experiment, LPS-mediated induction of cyclooxygenase-2, as well as PGE₂ biosynthesis, in macrophages in RAW 264.7 cells was blocked by as little as 50 μM of 3-phthalimido-2,6-dioxopiperidine. It appears, however, that LPS-enhanced cyclooxy-genase-2 transcription is not itself effected by the amide or imide. That is, the amide or imide has no effect on the induction of cyclooxygenase-2 by LPS. On the other hand, the amide or imide enhances the degradation of cyclooxygenase-2 messenger RNA. Consequently while not wishing to be bound by any theory, it appears the inhibitory effect of the amide or imide operates on the activity of cyclooxygenase-2 by some post-transcriptional mechanism.

The term alkyl denotes a univalent saturated branched or straight hydrocarbon chain containing from 1 to 6 carbon atoms. Representative of such alkyl groups are methyl, ethyl, propyl, isopropyl, butyl, isobutyl, sec-butyl, tert-butyl, pentyl, isopentyl, neopentyl, tert-pentyl, hexyl, and isohexyl.

Alkenyl denotes a univalent branched or straight hydrocarbon chain containing from 2 to 6 carbon atoms and an olefinic double bond. Typical alkenyl groups include vinyl, allyl, but-2-enyl, but-3-enyl, and the like.

Representative species include 3-phthalimido-2,6-dioxopiperidine, 1-allyl-3-phthalimido-2,6-dioxopiperidine, 1-ethyl-3-phthalimido-2,6-dioxopiperidine, 1-phenyl-3-phthalimido-2,6-dioxopiperidine, 1-benzyl-3-phthalimido-2,6-dioxopiperidine, 3-succimido-2,6-dioxopiperidine, and 1-allyl-3-succimido-2,6-dioxopiperidine. The preferred compound is 3-phthalimido-2,6-dioxopiperidine, also known as thalidomide.

The amides or imides utilized in the present invention are known and can be prepared by conventional techniques, as for example, set forth in the above cross-referenced patents and applications.

The amide or imide is preferably administered orally. Oral dosage forms include tablets, capsules, dragees, and similar shaped, compressed pharmaceutical forms containing from 1 to 100 mg of drug per unit dosage. Mixtures containing from 20 to 100 mg/mL can be formulated for parenteral administration which includes intramuscular, intrathecal, intravenous and intra-arterial routes of administration. Rectal administration can be effected through the use of suppositories formulated from conventional carriers such as cocoa butter.

Pharmaceutical compositions thus comprise the amide or imide associated with at least one pharmaceutically acceptable carrier, diluent or excipient. In preparing such compositions, thalidomide is usually mixed with or diluted by an excipient or enclosed within such a carrier which can be in the form of a capsule or sachet. When the excipient serves as a diluent, it may be a solid, semi-solid, or liquid material which acts as a vehicle, carrier, or medium for the active ingredient. Thus, the compositions can be in the form of tablets, pills, powders, elixirs, suspensions, emulsions, solutions, syrups, soft and hard gelatin capsules, suppositories, sterile injectable solutions and sterile packaged powders. Examples of suitable excipients include lactose, dextrose, sucrose, sorbitol, mannitol starch, gum acacia, calcium silicate, microcrystalline cellulose, polyvinylpyrrolidinone polyvinylpyrrolidone, cellulose, water, syrup, and methyl cellulose, the formulations can additionally include lubricating agents such as talc, magnesium stearate and mineral oil, wetting agents, emulsifying and suspending agents, preserving agents such as methyl- and propylhydroxybenzoates, sweetening agents or flavoring agents.

The amide or imide compositions preferably are formulated in unit dosage form, meaning physically discrete units suitable as a unitary dosage, or a predetermined fraction of a unitary dose to be administered in a single or multiple dosage regimen to human subjects and other mammals, each unit containing a predetermined quantity of active material calculated to produce the desired therapeutic effect in association with a suitable pharmaceutical excipient. The compositions can be formulated so as to provide an immediate, sustained or delayed release of active ingredient after administration to the patient by employing procedures well known in the art.

The amide or imide may possess a center of chirality and in such cases can exist as optical isomers. Both the chirally pure (R)- and (S)-isomers as well as mixtures (including but not limited to racemic mixtures) of these isomers, are within the scope of the present invention. Mixtures can be used as such or can be separated into their individual isomers mechanically as by chromatography using a chiral absorbent. Alternatively, the individual isomers can be prepared in chiral form or separated chemically.

The dosage employed must be carefully titrated to the patient considering his or her, weight, severity of the condition, and clinical profile. Typically the amount administered will be sufficient to produce a blood level of at least 0.01 μg/mL, preferably at least about 0.1 μg/mL. Thus the total blood volume in an average human (body weight 70 kg) is about 5 liters, so that an effective dose should provide a minimum of about 0.5 mg but can be as high as about 500 mg. Even higher doses may be required when the gut is inflamed, as it is in graft versus host disease and HIV infection. It also is known that some patients are susceptible to induced neuropathy and may require lower doses. Clinical experience may suggest doses from as low as 50 mg three times a week to as high as several grams per day but, as noted, the actual decision as to dosage must be made by the attending physician.

The following examples will serve to further typify the nature of the invention but should not be construed as a limitation on the scope thereof which is defined solely by the appended claims.

### EXAMPLE 1

Tablets, each containing 50 mg of 3-phthalimido-2,6-dioxopiperidine, can be prepared in the following manner:

Ingredients (for 1000 tablets)

| | |
|---|---|
| 3-phthalimido-2,6-dioxopiperidine | 50.0 g |
| lactose | 50.7 g |
| wheat starch | 7.5 g |
| polyethylene glycol 6000 | 5.0 g |
| talc | 5.0 g |
| magnesium stearate | 1.8 g |
| demineralized water | q.s. |

The solid ingredients are first forced through a sieve 25 of 0.6 mm mesh width. The active imide ingredient, the lactose, the talc, the magnesium stearate and half of the starch then are mixed. The other half of the starch is suspended in 40 ml of water and this suspension is added to a boiling solution of the polyethylene glycol in 100 ml of water. The resulting paste is added to the pulverulent substances and the mixture is granulated, if necessary with the addition of water. The granulate is dried overnight at 35° C., forced through a sieve of 1.2 mm mesh width and compressed to form tablets of approximately 6 mm diameter which are concave on both sides.

### EXAMPLE 2

Tablets, each containing 100 mg of 1-allyl-3-phthal-imido-2,6-dioxopiperidine, can be prepared in the following manner:

Ingredients (for 1000 tablets)

| | |
|---|---|
| 1-allyl-3-phthalimido-2,6- | 100.0 g |
| dioxopiperidine | |
| lactose | 100.0 g |
| wheat starch | 47.0 g |
| magnesium stearate | 3.0 g |

All the solid ingredients are first forced through a sieve of 0.6 mm mesh width. The active imide ingredient, the lactose, the magnesium stearate and half of the starch then are mixed. The other half of the starch is suspended in 40 ml of water and this suspension is added to 100 ml of boiling water. The resulting paste is added to the pulveru20 lent substances and the mixture is granulated, if necessary with the addition of water. The granulate is dried overnight at 35° C., forced through a sieve of 1.2 mm mesh width and compressed to form tablets of approximately 6 mm diameter which are concave on both sides.

### EXAMPLE 3

Tablets, each containing 10 mg of 3-succimido-2,6-dioxopiperidine, can be prepared in the following manner:

Ingredients (for 1000 tablets)

| | | |
|---|---|---|
| 3-succimido-2,6-dioxopiperidine | 10.0 | g |
| lactose | 328.5 | g |
| corn starch | 17.5 | g |
| 3-succimido-2,6-dioxopiperidine | 10.0 | g |
| lactose | 328.5 | g |
| corn starch | 17.5 | g |
| polyethylene glycol 6000 | S.0 | g |
| talc | 25.0 | g |
| magnesium stearate | 4.0 | g |

| | |
|---|---|
| demineralized water | q.s. |

The solid ingredients are first forced through a sieve of 0.6 mm mesh width. Then the 3-succimido-2,6-dioxopiperidine, lactose, talc, magnesium stearate and half of the starch are intimately mixed. The other half of the starch is suspended in 65 ml of water and this suspension is added to a boiling solution of the polyethylene glycol in 260 ml of water. The resulting paste is added to the pulverulent substances, and the whole is mixed and granulated, if necessary with the addition of water. The granulate is dried overnight at 35° C., forced through a sieve of 1.2 mm mesh width and compressed to form tablets of approximately 10 mm diameter which are concave on both sides and have a breaking notch on the upper side.

### EXAMPLE 4

Gelatin dry-filled capsules, each containing 50 mg of 3-phthalimido-2,6-dioxopiperidine, can be prepared in the following manner:

Ingredients (for 1000 capsules)

| | | |
|---|---|---|
| 3-phthalimido-2,6-dioxopiperidine | 50.0 | g |
| Lactose | 8.O | g |

The sodium lauryl sulphate is sieved into the 3-phthalimido-2,6-dioxopiperidine through a sieve of 0.2 mm mesh through a sieve of 0.9 mm mesh width and the whole is again intimately mixed for 10 minutes. Finally, the magnesium stearate is added through a sieve of 0.8 mm width and, after mixing for a further 3 minutes, the mixture is introduced in portions of 140 mg each into size 0 (elongated) gelatin dry-fill capsules.

### EXAMPLE 5

A 0.2% injection or infusion solution can be prepared, for example, in the following manner:

| | | |
|---|---|---|
| 3-phthalimido-2,6-dioxopiperidine | 5.0 | g |
| sodium chloride | 22.5 | g |
| phosphate buffer pH 7.4 | 300.0 | g |
| demineralized water to | 2500.0 | mL |

The active imide ingredient is dissolved in 1000 ml of water and filtered through a microfilter. The buffer solution is added and the whole is made up to 2500 ml with water. To prepare dosage unit forms, portions of 1.0 or 2.5 mL each are introduced into glass ampoules (each containing respectively 2.0 or 5.0 mg of imide).

What is claimed is:

1. A method of treating inflammatory disease caused or exacerbated by increased activity of cyclooxygenase-2, comprising administering to a mammal in need thereof an effective amount of an amide or imide of the formula: 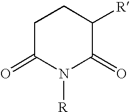
in which R is hydrogen, alkyl of 1 to 6 carbon atoms, alkenyl of 2 to 6 carbon atoms, morpholinomethyl, phenyl, or benzyl, and R′ is: 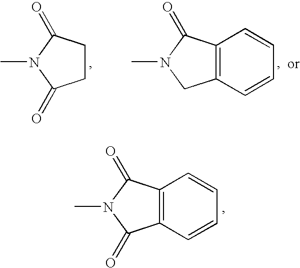
such that prostaglandin biosynthesis is inhibited; with the proviso that wherein R is H, R′ is not

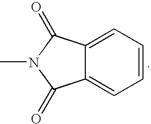

2. A method according to claim 1, wherein said amide or imide has an asymmetric center in substantially the (S) isomer.

3. A method according to claim 1, wherein said amide or imide has an asymmetric center in substantially the (R) isomer.

4. A method according to claim 1, wherein said amide or imide has an asymmetric center in an unequal mixture of the (S) and (R) isomers.

5. A method of inhibiting angiogenesis caused or exacerbated by increased activity of cyclooxygenase-2, comprising administering to a mammal in need thereof an effective amount of an amide or imide of the formula: 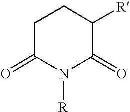
in which R is hydrogen, alkyl of 1 to 6 carbon atoms, alkenyl of 2 to 6 carbon atoms, morpholinomethyl, phenyl, or benzyl, and R′ is: 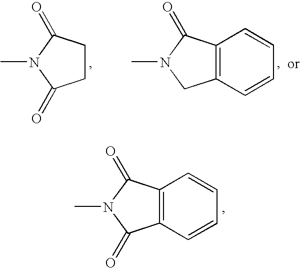
such that prostaglandin biosynthesis is inhibited; with the proviso that wherein R is H, R′ is not

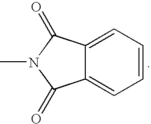

6. A method of inhibiting cell proliferation according to claim 5.

7. A method of inhibiting tumor growth according to claim 5.

8. A method according to claim 5, wherein said amide or imide has an asymmetric center in substantially the (S) isomer.

9. A method according to claim 5, wherein said amide or imide has an asymmetric center in substantially the (R) isomer.

10. A method according to claim 5, wherein said amide or imide has an asymmetric center in an unequal mixture of the (S) and (R) isomers.

11. A method of treating cancer caused or exacerbated by increased activity of cyclooxygenase-2, comprising administering to a mammal in need thereof an effective amount of an amide or imide of the formula: 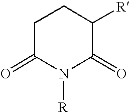
in which R is hydrogen, alkyl of 1 to 6 carbon atoms, alkenyl of 2 to 6 carbon atoms, morpholinomethyl, phenyl, or benzyl, and R′ is: 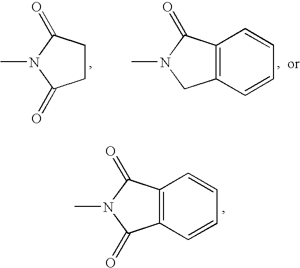
such that prostaglandin biosynthesis is inhibited; with the proviso that wherein R is H, R′ is not

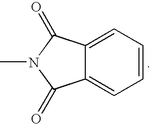

12. A method according to claim 11, wherein said cancer is selected from the group consisting of lung cancer, colorectal cancer, breast cancer, prostate cancer, bladder cancer, pancreatic cancer, ovarian cancer, leukemia, brain cancer, melanoma, lymphoma, erythroleukemia, uterine cancer and head and neck cancer.

13. A method according to claim 11, wherein said amide or imide has an asymmetric center in substantially the (S) isomer.

14. A method according to claim 11, wherein said amide or imide has an asymmetric center in substantially the (R) isomer.

15. A method according to claim 11, wherein said amide or imide has an asymmetric center in an unequal mixture of the (S) and (R) isomers.
